(19) United States
(12) Patent Application Publication
(10) Pub. No.: US 2025/0169800 A1
(43) Pub. Date: May 29, 2025

(54) SYSTEM FOR MINIMALLY INVASIVE EXTRACTION OF INTERSTITIAL FLUID

(71) Applicant: ISF Labs Inc., San Francisco, CA (US)

(72) Inventors: Samira Darvishi, San Francisco, CA (US); Anu Thubagere, San Francisco, CA (US)

(21) Appl. No.: 18/784,830

(22) Filed: Jul. 25, 2024

Related U.S. Application Data
(60) Provisional application No. 63/528,880, filed on Jul. 25, 2023.

(51) Int. Cl. A61B 10/00

(52) U.S. Cl. CPC A61B 10/0045; A61B 2010/008

(57) ABSTRACT

One variation of a vial assembly includes: a vial configured to collect interstitial fluid; a collar coupled to the vial and including a set of external retention features configured to locate a set of internal retention features of a base assembly; a needle casing coupled to the collar and enclosing the vial; a needle extending from the needle casing; and a bumper arranged on the needle casing opposite the vial, defining a needle aperture seated around and offset from the needle, and configured to apply a force to a skin surface of a patient, circumferentially about the needle, to create local hydrostatic pressure under the skin surface to drive interstitial fluid from below the skin surface, though the needle, and into the vial.

### CROSS-REFERENCE TO RELATED APPLICATIONS

This application claims the benefit of U.S. Provisional Application No. 63/528,880, filed on 25 Jul. 2023, which is incorporated in its entirety by this reference.

### TECHNICAL FIELD

This invention relates generally to the field of medical devices and more specifically to a new and useful system for extracting interstitial fluid in the field of medical devices.

### BRIEF DESCRIPTION OF THE FIGURES

FIG. 1 is a schematic representation of a system;

FIG. 2 is a flowchart representation of one variation of the system;

FIG. 3 is a flowchart representation of one variation of the system;

FIG. 4 is a schematic representation of one variation of the system;

FIG. 5 is a schematic representation of one variation of the system;

FIG. 6 is a flowchart representation of one variation of the system; and

FIG. 7 is a flowchart representation of one variation of the system.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. SYSTEM

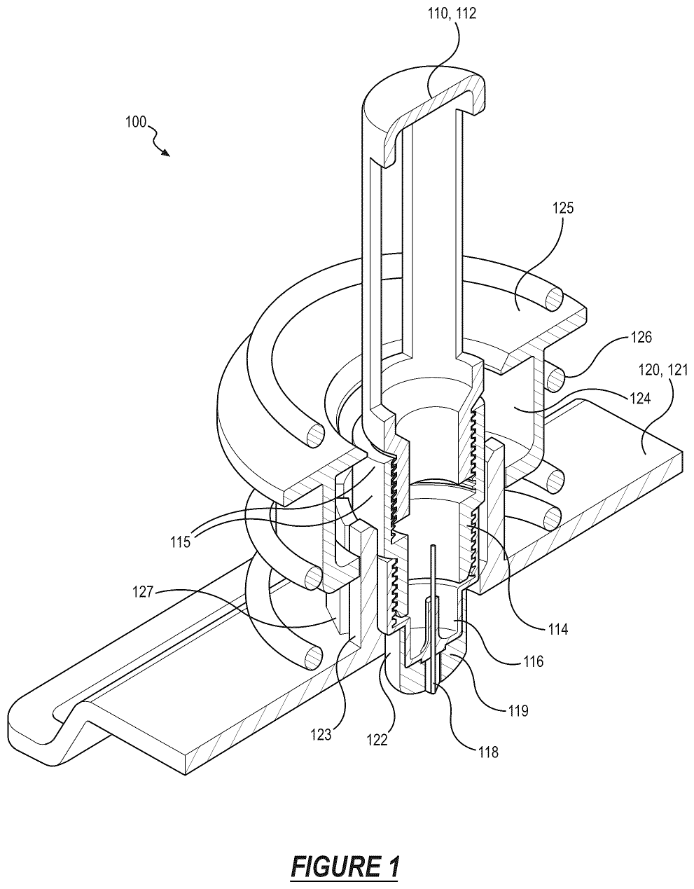

As shown in FIGS. 1-7, a system includes: a vial assembly 110; and/or a base assembly 120.

The vial assembly 110 includes: a vial 112 configured to collect interstitial fluid; a collar 114 coupled to the vial 112 and including a set of external retention features 115; a needle casing 116 coupled to the collar 114 and enclosing the vial 112; a needle 118 extending from the needle casing 116; and a bumper 119 arranged on the needle casing 116 opposite the vial 112 and defining a needle aperture seated around and offset from the needle 118.

The base assembly 120 includes: a base defining a base aperture configured to receive the vial assembly 110 and a column 123 extending above the base 121 aperture and including a set of internal retention features 124 configured to locate the set of external retention features 115 and configured to locate over a skin surface of a patient; a lock ring 125 operable in an unlocked position and a locked position, the lock ring 125 decoupled from the set of external retention features 115 in the unlocked position and depressing the set of internal retention features 124 inwardly toward the aperture, to engage the set of external retention features 115 of the vial assembly 110 and to retain the vial assembly 110 in a particular depth position in a range of depth positions within the base 121 aperture, in the locked position. The base assembly 120 is configured to depress the bumper 119 of the vial assembly 110 against the skin surface of the patient at a force corresponding to the particular depth position of the vial assembly 110 within the base 121 aperture. In this system, the bumper 119 applies the force to the skin surface of the patient, circumferentially about the needle 118, to create local hydrostatic pressure under the skin surface to drive interstitial fluid from below the skin surface, though the needle 118, and into the vial 112.

### 1.1 Variation: Base Assembly

In one variation, the system includes a base assembly 120 including a base: defining a base aperture configured to receive a vial assembly 110 and a column 123 extending above the base 121 aperture and including a set of internal retention features 124 configured to locate a set of external retention features 115 of the vial assembly 110; and configured to locate over a skin surface of a patient. The base assembly 120 also includes a lock ring 125: operable in an unlocked position and a locked position; decoupled from the set of external retention features 115 in the unlocked position; and depressing the internal retention features 124 inwardly toward the aperture, to engage the set of external retention features 115 of the vial assembly 110 and to retain the vial assembly 110 in a particular depth position in a range of depth positions within the base 121 aperture, in the locked position. In this variation, the base assembly 120 is configured to: depress a bumper 119 of the vial assembly 110 against the skin surface of the patient at a force corresponding to the particular depth position of the vial assembly 110 within the base 121 aperture, the bumper 119 of the vial assembly 110; and apply the force to the skin surface of the patient, circumferentially about the needle 118, to create local hydrostatic pressure under the skin surface to drive interstitial fluid from below the skin surface, though the needle 118, and into the vial 112.

### 1.2 Variation: Vial Assembly

In another variation, the system includes a vial assembly 110 including: a vial 112 configured to collect interstitial fluid; a collar 114 coupled to the vial 112 and including a set of external retention features 115 configured to locate a set of internal retention features 124 of a base assembly 120; a needle casing 116 coupled to the collar 114 and enclosing the vial 112; a needle 118 extending from the needle casing 116; and a bumper 119 arranged on the needle casing 116 opposite the vial 112, defining a needle aperture seated around and offset from the needle 118, and configured to apply a force to a skin surface of a patient, circumferentially about the needle 118, to create local hydrostatic pressure under the skin surface to drive interstitial fluid from below the skin surface, though the needle 118, and into the vial 112.

### 2. APPLICATIONS

Generally, the system includes a disposable vial assembly 110 including: a vial 112 configured to collect interstitial fluid; a collar 114 coupled to the vial 112 and including a set of external retention features 115 (such as a set of annular ribs extending radially from the collar 114); a needle casing 116 transiently coupled to the collar 114 and enclosing the vial 112; a needle 118 extending from the needle casing 116; and a bumper 119 arranged on the needle casing 116 opposite the vial 112, and seated around and offset from the needle 118 such that the needle 118 extends from the needle casing 116, through the bumper 119, to a surface of skin of a patient. The system further includes a reusable base assembly 120 including: a base defining slots for an elastic strap; an aperture centered with and coaxial to the base 121; a column 123 extending upward from the aperture including a set of internal retention features 124 and configured to receive the vial assembly 110; a lock ring 125 operable in locked and unlocked positions and biased toward the unlocked position by the spring 126; and a ratchet mechanism 127 located on the base 121 and configured to selectively retain the lock ring 125 in the locked position.

In particular, in the locked position, the lock ring 125 drives the internal retention features 124 of the base 121 inwardly to engage external retention features 115 of the collar 114, which thus retain the vial assembly 110 in a consistent vertical position (and pitch orientation) within the base 121 aperture. Accordingly, the base assembly 120 maintains contact between the bumper 119 of the vial assembly 110 and the patient's skin with a consistent force or pressure as the needle 118—thus inserted into the patient's dermis-draws interstitial fluid upwardly into the vial 112 as a result of hydrostatic pressure within the dermis, which may result from local application of the bumper 119 around the needle 118. In the unlocked position the lock ring 125 releases the internal retention features 124 of the base 121, which thus expand outwardly to release the external retention features 115 of the collar 114, thereby releasing the vial assembly 110 from the base assembly 120. Accordingly, the user: may depress the via further toward the patient's skin surface—thereby increasing the force applied by the bumper 119, increasing local hydrostatic pressure, and possible increasing flow rate of interstitial fluid into the vial 112—before (again) transitioning the lock ring 125 into the locked position; or otherwise remove the vial assembly 110 from the base assembly 120 upon collecting an interstitial fluid sample within the vial 112.

More specifically, when arranged over a region of skin on a patient, the bumper 119 depresses against the surface of the skin and exerts a local force on the skin, thereby causing a first subsection of the skin around the needle 118 to depress inwardly and increase local hydrostatic pressure within an interstitial volume between cells in the dermis adjacent the needle 118. A resulting pressure difference between local hydrostatic pressure within the dermis proximal the needle 118 and ambient pressure (or pressure within the vial assembly 110) may thus cause interstitial fluid to flow from the dermis, through the needle 118, and into the vial 112 without additional pumps, plungers, or other moving parts. The base assembly 120 is thus configured to receive, locate, and retain the vial assembly 110 over the skin surface while maintaining consistent application of force or pressure by the vial assembly 110 onto the skin surface such that elevated local hydrostatic pressure persists proximal the needle 118 during collection of interstitial fluid. The lock ring 125 thus functions to manipulate internal retention features 124 on the base 121 to selectively (i.e., transiently) retain and release the vial assembly 110.

For example, the system can extract interstitial fluid by: locating a distal end of a needle 118 in a patient's dermis at a consistent depth between 0.040″ and 0.060″ below the surface of the skin and thus avoiding contact between the needle 118 and capillary beds within the lower dermis (and thereby avoiding contamination of an interstitial fluid by blood cells released by a lysed capillary bed); and applying pressure to a local region of a the patient's skin via the bumper 119, which applies a circumferential force to the skin surface around the needle 118, which increases local hydrostatic pressure within an interstitial volume within the dermis proximal the needle 118, which induces flow of interstitial fluid from the dermis, through the needle 118, and into the vial 112. The vial assembly 110 in includes the needle 118, vial 112, bumper 119, and external retention features 115 for setting and maintain the force applied by the bumper 119 to the skin surface. The base assembly 120 includes a base configured to locate (e.g., strap) onto the patient's body (e.g., arm, leg, buttocks, or abdomen), a base aperture that receives the vial assembly 110, and internal retention features 124 and the lock that cooperate to selectively retain the vial assembly 110 in a vertical position selected by a user.

Furthermore, internal retention features 124 (e.g., “finger” or “cam followers” arranged on living hinges extending upwardly from the base 121 proximal the base 121 aperture and the column 123) can be configured to follow (e.g., “lightly run over”) and vibrate against the external retention features 115 of the collar 114 of the vial assembly 110 when the lock ring 125 occupies the unlocked position and the vial assembly 110 is inserted into or withdrawn from the aperture. The internal retention features 124 of the base assembly 120 can thus cooperate with the external retention features 115 of the vial assembly 110 to output haptic feedback to the user to indicate changes in vertical position of the vial assembly 110 within the base assembly 120—and thus changes in force applied by the bumper 119 to the skin surface of the patient.

### 2.1 Interstitial Fluid Sample Contamination by Local Pain Response

Generally, presence and certain concentrations of compounds within interstitial fluid may indicate underlying diseases and conditions, such as: diabetes; bacterial infections; cancer; heart disease; and/or inflammatory diseases. However, painful collection of interstitial fluid may contaminate the interstitial fluid sample with additional compounds released by the body at the site of needle insertion (i.e., the “sample site”), such as: prostaglandins; bradykinins; neuropeptides; histamines; cytokines; serotonin; hydrogen ions; and/or leukotrienes. In particular, the presence of such pain and/or inflammatory markers within an interstitial fluid sample: may contaminate the sample; and thus yield ‘noisy’ or unrepresentative sample analysis results. For example, increased immune cell activation (such as substance P, anti-inflammatory cytokines, and/or macrophages) resulting from a local pain response may mask (or alter) the presence of specific biomarkers (e.g., electrolyte content, elevated white blood cell count due to an infection, heart function-related markers) associated with an underlying disease or condition, thereby preventing diagnosis or inhibiting management of such a disease or condition based on interstitial fluid test results.

For example, when creating a blister at a sample site on a patient's body, the patient's body may respond to this painful input by releasing (some of) these compounds locally into interstitial fluid near the sample site such that these compounds collect in the blister in concentrations greater than elsewhere in the patient's body. Accordingly, this sample may not be representative of interstitial fluid throughout the entire body of the patient. (Because interstitial fluid may not be drawn from this blister until hours or days later, compounds in this interstitial fluid sample may also decay or may be reabsorbed into the patient's body such that concentrations of the compounds are further less representative of interstitial fluid elsewhere in the patient's body at time of sample collection.) Similarly, a pain response during insertion of the needle 118 into a patient's body at a sample site—such as due to penetration into a blood vessel or rupture of a capillary—may similarly trigger immediate changes in concentration of these compounds within the interstitial fluid sample such that the sample is not representative of interstitial fluid elsewhere in the patient's body. Therefore, local pain at the sample site may trigger an immediate inflammatory and/or hormonal response, which may result in local release of compounds into interstitial fluid near the sample site. Presence of these compounds may: decrease a signal-to-noise ratio in the sample; and/or increase concentration of a target compound in the sample such that test results from the sample are not accurate or useful in diagnosing or managing a disease. Thus, by creating a pain response at the sample site, test results derived from the interstitial fluid sample: may be biased toward compounds released by the human body responsive to local pain input; and may therefore not be representative of the global contents of bodily interstitial fluid.

Therefore, a contaminated sample: may be unrepresentative of global interstitial fluid; obscure concentrations and/or ratios of biomarkers indicative of an underlying disease; inhibit disambiguation of signals (i.e., compounds, compound concentrations) resulting from local pain from signals resulting from an underlying acute condition; and thus inhibit accurate analysis of the sample.

### 2.2 Local Pain Response Avoidance

Conversely, the system can enable immediate and pain-free collection of interstitial fluid from a patient. Accordingly, the interstitial fluid sample collected by the system: may be uncontaminated by compounds released by the body at the sample site; and may include combinations and concentrations of compounds than are more representative of presence and concentrations of compounds throughout the patient's entire body. More specifically, the system can collect an interstitial fluid sample containing few or no additional compounds resulting from a local pain and/or inflammatory response by eliminating a perceived pain response at the sample site.

In particular, the system can enable extraction of an interstitial fluid sample exhibiting no or minimal contamination by: inserting a needle 118 into the dermis, and avoiding capillary beds, to thus draw interstitial fluid directly from an interstitial volume between collagen bundles and lining cells of the dermis via local hydrostatic pressure, thereby avoiding large or bulky vacuum systems that may cause discomfort, pain, and inadvertent advancement of the needle 118 deeper toward pain receptors in the skin; and by collecting the interstitial fluid directly (e.g., rapidly, immediately) from the dermis (i.e., rather than from a blister).

The system can thus collect an interstitial fluid sample that: is uncontaminated (or not “clouded”) by compounds released responsive to local pain signals; is representative of current global interstitial fluid throughout the patient's body; enables greater accuracy in analyte processing; and accurately reveals presence and concentrations of biomarkers indicative of underlying disease.

Therefore, the system enables immediate, rapid, and painless (or low-pain) collection of interstitial fluid that may exhibit no or minimal contamination by compounds resulting from nociceptive response and thus may support more accurate analysis of interstitial fluid and disease diagnosis or management based on interstitial fluid test results.

### 3. EXAMPLE

Generally, the system can be used in a healthcare or research setting by a user or a healthcare professional to collect interstitial fluid.

For example, a healthcare professional may: secure the base 121 to a patient (e.g., on the forearm, calf, upper arm, stomach), such as via a strap 130 looped through each end of the base 121; insert the vial assembly 110 to the base 121 until the patient notifies the health care professional that the tip of the needle 118 is touching the top of the skin, and not breaching the skin; and, when the patient is ready, the healthcare professional (or the patient) may depress the top of the lock ring 125, thus inserting the needle 118 into shallow layers of the dermis (e.g., between 0.040″ and 0.060″ below the surface of the skin), thus avoiding capillary beds located deeper in the dermis and generating localized hydrostatic pressure driving interstitial fluid (uncontaminated by blood) into the vial 112.

More specifically, the healthcare professional may: select a region of the body defining a soft tissue, such as the forearm or upper arm; secure the base 121 to the region of the body by threading a piece of elastic through loops on the side of the base 121, thus stabilizing the base 121 to the patient; insert the vial assembly 110 into the base 121 via the set of external retention features 115, such as ridges that click the vial 112 into place, until the patient provides feedback, alerting the healthcare professional that the needle 118 is touching the skin, and the bumper 119 is applying a local pressure to the surface of the skin proximal the needle 118; and depress the top of the base 121, such as on the lock ring 125. In response to the user depressing the lock ring 125: the spring 126 compresses, driving the vial assembly 110 downward toward the surface of the skin, thus decoupling the set of retention features to enable the vertical shift, and the system drives the needle 118 into the skin, thus inserting the needle 118 into the dermis (e.g., at a depth of between 0.040″ and 0.060″ below the surface of the skin). The local hydrostatic pressure from the bumper 119 creates a pressure difference between the interstitial fluid and the vial 112, thus driving interstitial fluid into the lower-pressure vial 112.

Then, after the vial 112 reflects an adequate collection of interstitial fluid, the healthcare professional may: lift the vial assembly 110, thus removing the needle 118 from the patient's skin; remove the vial 112 from the vial assembly 110; transport the vial 112 to sample handling and analysis; dispose of the needle 118, bumper 119, collar 114, and needle casing 116; and remove the base assembly 120 from the patient and sanitize for reuse.

Therefore, the system can enable: pain-free collection of interstitial fluid substantially uncontaminated by compounds that may rapidly enter interstitial fluid near sights of local pain responses; rapid collection of interstitial fluid; and collection of interstitial fluid without pumps or electromechanical systems.

### 4. VIAL ASSEMBLY

Generally, the vial assembly 110 includes: a vial 112 configured to collect interstitial fluid; a collar 114 coupled to the vial 112 and including a set of external retention features 115; a needle casing 116 coupled to the collar 114 and enclosing the vial 112; a needle 118 extending from the needle casing 116; and a bumper 119 arranged on the needle casing 116 opposite the vial 112 and defining a needle aperture seated around and offset from the needle 118.

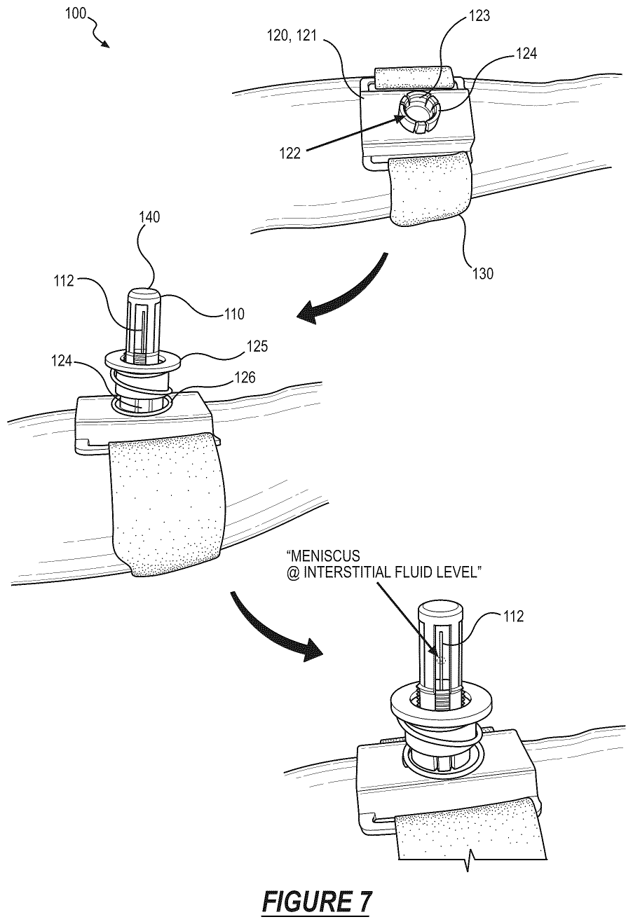

More specifically, as shown in FIGS. 1 and 7, the vial assembly 110 is configured to transiently locate within the aperture defined by the column 123 of the base 121 and to collect a sample of interstitial fluid from a patient. The vial assembly 110 includes: a vial 112 configured to receive and store interstitial fluid; a collar 114 coupled to the vial 112 and including a set of external retention features 115 (e.g., a helical thread, a set of annular ribs) configured to mate with internal retention features 124 of the base 121 to retain the vial 112 within the base 121 aperture of the base 121; and a needle 118 assembly including a needle casing 116 configured to couple to (e.g., screw onto) the collar 114 opposite the vial 112 and a needle 118 extending from the needle casing 116. The vial assembly 110 further includes a bumper 119: configured to install (or “seat”) over the needle casing 116 (e.g., via an interference fit); including a needle aperture configured to locate around the needle 118; and configured to depress against the skin surface of a patient in order to increase local hydrostatic pressure within the dermis, which may drive interstitial fluid from the dermis, through the needle 118, and into the vial 112 without need for additional pumps, plungers, or other hardware or subsystems. The bumper 119 also: defines a distal end—that forms a first point of contact of the bumper 119 with the patient's skin surface—offset behind the distal end of the needle 118 by a target or nominal insertion distance of the needle 118 into the patient's skin (e.g., between 0.040″ and 0.060″); and includes an elastic material such that the bumper 119 deforms when depressed against the patient's skin surface, thereby enabling a user to increase the insertion distance of the needle 118 and concurrently increasing local hydrostatic pressure within the dermis by depressing the vial assembly 110 against the patient with greater force.

In particular, the base assembly 120 locates the vial assembly 110 over the sample site. The vial assembly 110 locates the needle 118 over the sample site. The bumper 119 both sets the insertion distance of the needle 118 into the patient's skin and applies local pressure to the patient's skin in order to increase local hydrostatic pressure within the dermis.

### 4.1 Bumper

In one implementation, the bumper 119 defines: an attachment surface (e.g., an undersized counterbore) configured to assemble onto a distal end of the needle casing 116 (e.g., via a 0.005″ interference fit); a bell-shaped region extending distally from the attachment surface; and an annular ridge arranged on a distal end of the bell-shaped region, encircling the needle aperture, offset above a distal end of the needle 118 and defining a first point of contact between the bumper 119 and the skin region.

Furthermore, the bumper 119: can include an elastomeric material of a durometer between Shore 40 and Shore 80; and can be configured to deform against the skin surface during further insertion of the vial assembly 110 into the base 121. More specifically, the bumper 119 can include an elastomeric material: configured to sit on the surface of the skin of the patient; and defining a elasticity and moderate hardness such that, upon coming into contact with the skin, the bumper 119 deforms against the patient's skin to both increase hydrostatic pressure around the sample site and enable small, controlled increases in depth of the needle 118 as the vial assembly 110 is depressed against the sample site with greater force, thereby avoiding excess insertion of the needle 118 into the patient's skin and risk of increased patient discomfort or pain.

Thus, the bumper 119: is configured to control the insertion distance of the needle 118 into the skin of the patient; and applies a local force to the skin surface at this sample site, thereby increasing local hydrostatic pressure such that, with the needle 118 concurrently inserted into the skin, a difference in pressure between the dermis at the sample site and pressure within the vial 112 (E.g., ambient air pressure) causes interstitial fluid to flow from the dermis, through the needle 118, and into the vial 112.

Furthermore, because the bumper 119 is elastomeric or otherwise configured to deform when applied against the patient's skin surface, elasticity of the bumper 119 may further reduce perception of pain when the via is driven and retained against the patient's skin, thereby further reducing risk of contamination of an interstitial fluid sample thus collected.

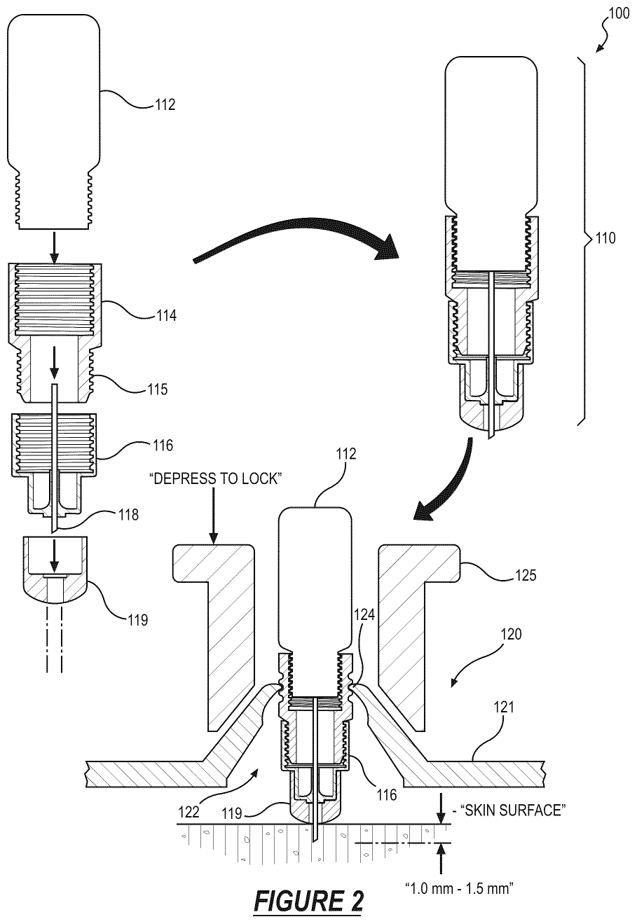

In one implementation shown in FIG. 2, the needle 118 includes a sterile hypodermic needle defining an outer diameter. The bumper 119: defines an internal counterbore configured to seat on the distal end of the needle casing 116 via a first interference fit; and defines the needle aperture coaxial with the internal counterbore. IN this implementation, the vial assembly 110 further includes a sterile sleeve 111: defining an inner diameter greater than the outer diameter of the needle 118; configured to transiently locate within the needle aperture of the bumper 119 via a second interference fit; configured to prevent contact between the bumper 119 and the needle 118 during assembly of the bumper 119 onto the needle casing 116; and removable from the bumper 119 to expose the needle 118 prior to insertion of the vial assembly 110 into the base 121 aperture of the base 121.

More specifically, in this variation, the system further includes a sterile sleeve 111 for isolating the bumper 119 from the needle 118 and preserving sterility of the needle 118 during assembly of the vial assembly 110 and prior to insertion of the needle 118 into the patient. For example, bumper 119 can be stored in sterile packaging with the sterile sleeve 111 (e.g., a sterile steel tube) inserted into the needle aperture of the bumper 119. Following assembly of the bumper 119 and sterile sleeve 111 onto the needle casing 116 and around the needle 118, a user may slide the sterile sleeve 111 off of the needle 118 and the bumper 119 to reveal the needle 118 just prior to insertion of the vial assembly 110 into the base assembly 120.

### 4.2 Collar External Retention Features

As shown in FIGS. 1, 2, 3, 4 and 6, the collar 114 includes the set of external retention features 115, such as including: a first annular rib arranged on the collar 114 and extending radially outwardly from collar 114; a second annular rib arranged on the collar 114, extending radially outwardly from the collar 114 and offset above the first annular rib; and a third annular rib arranged on the collar 114, extending radially outwardly from collar 114 and offset above the second annular rib. In this implementation, the lock ring 125 is configured to: compress the set of internal retention features 124 to locate an internal retention feature 124, in the set of internal retention features 124, between the first annular rib and the second annular rib to locate a distal end of the needle 118 at a first depth below the base 121; and compress the set of internal retention features 124 to locate the internal retention feature 124, in the set of internal retention features 124, between the second annular rib and the third annular rib to locate the distal end of the needle 118 at a second depth, less than the first depth, below the base 121.

More specifically, the collar 114 can include the set of external retention features 115 that from external mating features (e.g., annular ribs) configured to engage internal retention features 124 on the base assembly 120 when closed against the collar 114 by the lock ring 125. For example, the internal retention features 124 can each include a point configured to locate within lock against a valley formed by two adjacent annular ribs extending outwardly from the collar 114.

Furthermore, the collar 114 can include a column 123 of internal retention features 124 that cooperate to enable the vial assembly 110 to locate within the base assembly 120 over a range of vertical positions. For example, internal retention features 124 can include ten annular ribs on a vertical pitch of 0.030″, thus forming eight valleys configured to engage the internal retention features 124 of the base assembly 120 over a range of 0.240″ with 0.030″ steps.

Alternatively, rather than discrete annual ribs, the external retention features 115 can instead include a continuous external helical thread, such as a 60°-base thread characterized by a pitch of 0.030″. In this implementation, the internal retention features 124 can be located above the base 121 at different vertical positions corresponding to the pitch of the continuous external helical thread such that the internal retention features 124 locate the vial assembly 110 squarely in the base 121 aperture when engaging the continuous external helical thread on the collar 114. In particular, in this implementation, the set of external retention features 115 includes: an external helical thread configured to engage an internal helical thread of the column 123 to locate the vial assembly 110 over a range of depths in the column 123. In this implementation, the bumper 119 can be configured to: rotate (or pivot) about the needle casing 116 and the needle 118 while remaining in a fixed angular position on the skin surface of the patient as the external helical thread of the collar 114 engages the internal helical thread of the column 123, thereby reducing or eliminating twisting of the patient's skin and reducing or eliminating perception of pain at the sample site.

In one implementation, the collar 114 is formed of an injection-molded polymer and includes: internal threads configured to thread onto external threads of the vial 112; and external threads configured to thread onto internal threads of the needle casing 116. However, the collar 114 can be formed in any other way and in any other material.

### 4.3 Vial

In one implementation, the vial 112 includes a glass or polymer blow-molded or drawn container, such as including external threads configured to thread onto the collar 114 and forming a total internal volume of one milliliter.

The vial 112 can further includes a diaphragm seal 113 located across an opening (or “mouth”) on a distal end of the vial 112 and configured to seal the internal volume of vial 112. In this implementation, the needle 118 can also include a proximal end extending rearward from the needle casing 116 and configured to puncture the diaphragm seal 113 of the vial 112 during assembly of the needle casing 116 onto the collar 114. In this implementation, the needle 118 can puncture the diaphragm seal 113 at the distal end of the vial 112 to form a sterile seal between the needle 118 and the vial 112 such that interstitial fluid flowing through the needle 118 is collected by and fully contained within an internal volume formed by the vial 112, the diaphragm seal 113, and the needle 118, thereby avoiding contact between the interstitial fluid and the collar 114.

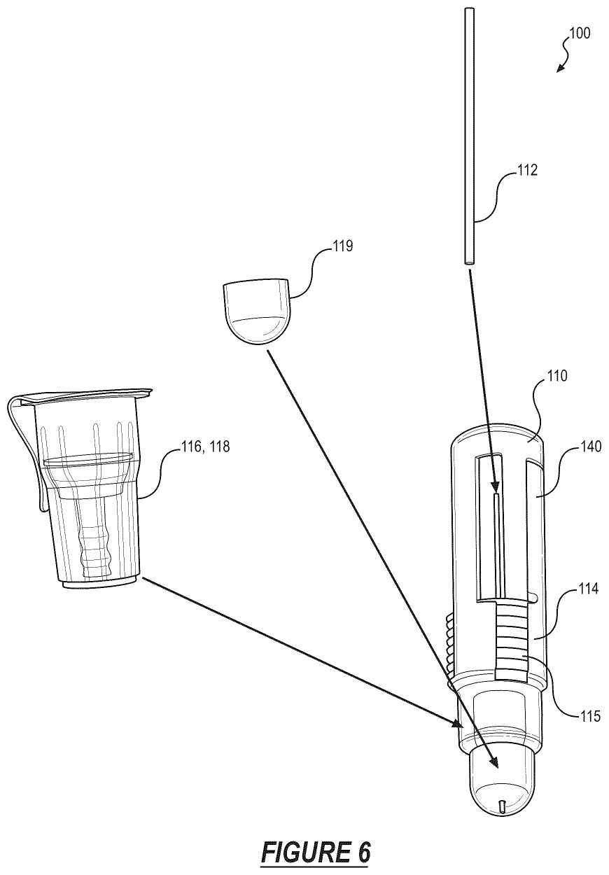

In one variation shown in FIGS. 6 and 7, the vial assembly 110 further includes a vial guard 140 extending from (i.e., contiguous with) or assembled over a proximal end of the collar 114 can configured to shield the vial 112 from damage or fracture.

### 5. BASE ASSEMBLY

Generally, the system includes a base assembly 120 including: a base defining a base aperture configured to receive the vial assembly 110 and a column 123 extending above the base 121 aperture and including a set of internal retention features 124 configured to locate the set of external retention features 115 of the collar 114 of the vial assembly 110, and configured to locate over a skin surface of a patient; a lock ring 125 operable in an unlocked position and a locked position, decoupled from the set of external retention features 115 in the unlocked position, and depressing the set of internal retention features 124 inwardly toward the aperture, to engage the set of external retention features 115 of the vial assembly 110 and to retain the vial assembly 110 in a particular depth position in a range of depth positions within the base 121 aperture, in the locked position. The base assembly 120 is configured to depress the bumper 119 of the vial assembly 110 against the skin surface of the patient at a force corresponding to the particular depth position of the vial assembly 110 within the base 121 aperture.

As shown in FIGS. 1 and 2, the base assembly 120 includes: a base defining an aperture centered on the base 121, and a column 123 extending upwardly from the base 121 through the aperture defining a set of internal retention features 124 configured to accept the vial assembly 110 by locating the set of external retention features 115 of the column 123. The base assembly 120 further includes a lock ring 125: configured to shift the system vertically toward the skin of the patient to maintain a consistent needle depth upon insertion and across uses; defining an unlocked position decoupled from the set of external retention features 115 such that the set of internal retention features 124 maintain a relaxed/flexible state to enable the vial assembly 110 to be inserted and the vertical position of the vial assembly 110 to be adjusted; and defining a locked position configured to secure the set of external retention features 115 on the collar 114 to the set of internal retention features 124 on the column 123 and thus locking the vial assembly 110 in place at the vertical position and normal to the surface of the skin.

### 5.1 Base

Generally, the base 121 defines an interior surface configured to contact a skin surface of a patient, such as an arm (e.g., a forearm), a leg (e.g., a thigh, a calf), a buttocks, or an abdomen.

In one implementation, the interior surface of the base 121 defines an inverted ‘V’-shape (or a “V-block”) in which each leg of the interior surface is configured to locate over a side of an extremity of a patient. More specifically, because the base 121 defines an interior Virtual-shaped surface, the base 121 can seat over pseudo-cylindrical extremities (i.e., arms, legs) of a range of sizes. Furthermore, the base of the V-shaped interior surface may be offset from an adjacent skin surface proportional to a size or effective diameter of the extremity on which the base assembly 120 is located; therefore the collar 114 can include a column 123 of external retention features 115 configured to engage the internal retention features 124 of the base assembly 120 over a range of vertical distances in order to accommodate positioning of the base 121 over extremities of different sizes.

For example, the base 121 can define: a flat surface; the base 121 aperture intersecting the flat surface; a first inclined surface rightward of the flat surface; and a second inclined surface leftward of the flat surface and cooperating with the first inclined surface to form an inverted ‘V’-shape. In this example, when the base 121 is located on a first small arm, such as a child's arm or a woman's arm, the base 121 locates flat surface and the base 121 aperture at a first offset distance above the patient's skin surface. Later, when the base 121 is located on a larger arm, such as an adult's arm or a man's arm, the base 121 locates the flat surface and the base 121 aperture at a second offset distance above the patient's skin surface greater than the first offset distance. Thus, though variation in arm size may result in location of the base 121 aperture over a range of offset distances from the skin surface of the patient, ‘V’-shape of the base 121 can: reliably locate the base 121 on extremities of many sizes and shapes. Additionally, the collar 114 of the vial assembly 110 defines the set of engagement features that cooperate with the base 121 to locate the vial assembly 110 over a range of vertical positions in order to accommodate the range of offset distances between the aperture and the skin surface for various extremities sizes and shapes.

Additionally or alternatively, the interior surface of the base 121 can define a curved surface, such that the curved surface configures to or mates with the shape of an extremity of a patient, such as the shape of a forearm and/or the shape of a calf.

However, the base 121 can define any other shape geometry and can interface with a region of a patient's body in any other way.

### 5.2 Spring+Lock Ring+Ratchet Mechanism

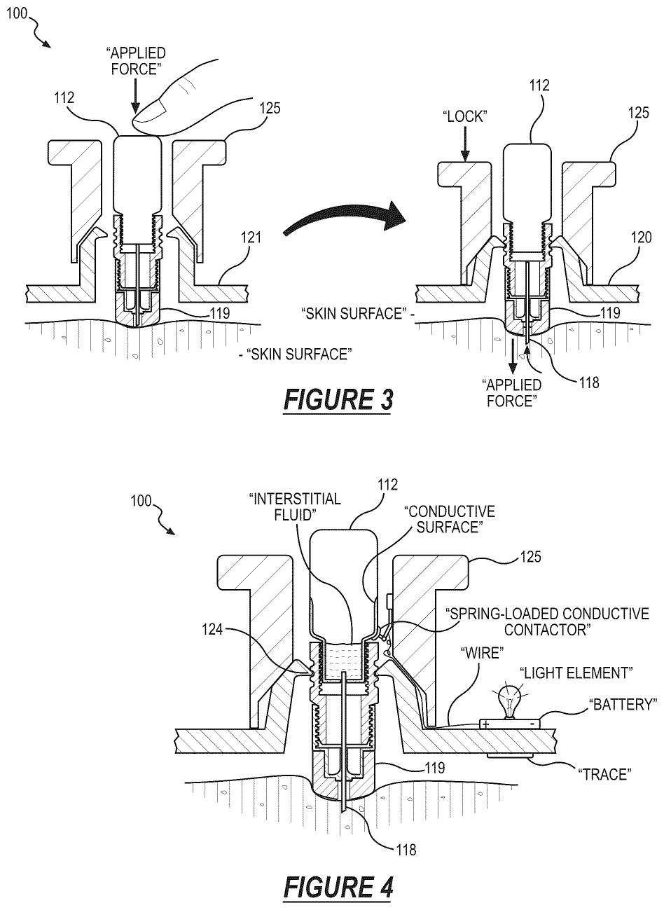

In one variation shown in FIG. 3, the system further includes: a spring 126 coupled to the lock ring 125 of the base assembly 120 and configured to bias the lock ring 125 into the unlocked position; and a ratchet mechanism 127 (e.g., a retractable pen mechanism including a rotating cam and a guide) arranged on the base 121 and that cooperates with the spring 126 to selectively retain the lock ring 125 in the locked position.

In one implementation, the spring 126 includes a coil spring arranged about and outside of the column 123. The lock ring 125 rides (or “runs”) vertically on the column 123 and includes a set of internal cam surfaces (or “ramps”) including upwardly toward the aperture and radially offset by 120° about the aperture. The column 123 includes a set of slots (or “reliefs”), such as three slots radially offset by 120° about the aperture. The internal retention features 124 includes a set of cams, each cam: extending upwardly from the base 121; occupying a slot in the column 123; including an internal point configured to mate with (or “locate in”) an external retention feature 115 on the collar 114; including an external cam surface configured to ride on a corresponding inclined cam surface on the lock ring 125; and including a living hinge proximal the base of the column 123.

In this implementation, the spring 126 is configured to: bias the lock ring 125 (upwardly) away from the base 121; and to return the lock ring 125 to the unlocked position responsive to release of the lock ring 125 by the ratchet mechanism 127. When the lock ring 125 occupying the locked position is depressed, the ratchet mechanism 127 releases the lock ring 125, and the spring 126 transitions the lock ring 125 into the unlocked position. Accordingly, the lock ring 125 withdraws from the internal retention features 124 on the base assembly 120 and enables the internal retention features 124 to expand outwardly, thereby releasing the vial assembly 110, such as to enable the user to remove the vial assembly 110 from the base assembly 120 or further depress the vial assembly 110 downward to increase pressure applied by the bumper 119 to the skin surface of the patient. Conversely, when the user depresses the lock ring 125—such as while concurrently depressing the vial assembly 110 downwardly against the patient's skin surface with a preferred force or pressure—the spring 126 yields as the internal cam surfaces on the lock ring 125 ride down corresponding external cam surfaces on the internal retention features 124 and thus drive the internal retention features 124 inwardly to engage and retain external retention features 115 on the vial assembly 110. The ratchet mechanism 127 further engages the lock ring 125 to retain the lock ring 125 in the locked position with the internal retention features 124 retaining the vial assembly 110 such that the base assembly 120 maintains application of (approximately) the preferred force or pressure of the bumper 119 on the patient's skin surface.

Furthermore, when the lock ring 125 is retracted into the unlocked position, the set of internal retention features 124 expand to release the external retention features 115. However, the internal retention features 124 can define points—configured to engage the external retention features 115—that fall on a circle of diameter (slightly) less than the outer diameter of the external retention features 115 when the lock ring 125 is unlocked. Accordingly, further insertion of retraction of the vial assembly 110 within the base 121 aperture may cause the internal retention features 124 to run along the external retention features 115, thereby product audible and/or haptic feedback to enable a user to more accurately perceive changes in position of the vial assembly 110 within the base 121 aperture.

### 5.3 Strap

In one variation shown in FIG. 7, the system further includes a strap 130: arranged on the base 121; and configured to retain the base 121 on an extremity of the patient, such as a leg or arm, with limited compliance (i.e., limited movement of the base assembly 120 over the skin surface). In one example, the base 121 is configured to locate the base 121 aperture and the vial assembly 110 over the forearm of the patient. In one implementation, the strap 130 includes an elastic cuff (or “band”) extending from opposite ends of the base 121 and cooperates with the base 121 to form a sleeve that slides over a hand and forearm to locate on an upper forearm or upper arm of a patient.

In another implementation, the strap 130 includes a hook-and-loop strap assembly extending from opposite ends of the base 121 and configured to transiently secure the base assembly 120 to an arm, a leg, a torso (e.g., to locate the vial assembly 110 over the patient's abdomen), or other region on a patient.

### 5.4 Manufacturing

In one implementation, the base 121 includes a polymer (or plastic) or composite structure, such as injection molded polycarbonate or 3D printed in nylon.

Alternatively, the base 121 include an metal structure, such as machined from aluminum or stainless steel billet, die-cast in aluminum, or stamped and formed in sheetmetal.

The lock ring 125 and ratchet mechanism 127 can be similar formed or fabricated in similar materials.

### 6. ASSEMBLY AND OPERATION

Generally, during use, the strap 130 retains the base 121 against the skin of the patient with the aperture of the base 121 located over the sample site on the patient's skin surface. The base 121 receives and locates the vial assembly 110 over the sample site. The lock ring 125 selectively transitions the internal retention features 124 on the base 121 toward the vial assembly 110 to maintain a vertical position of vial assembly 110 within the aperture and thus control a force applied by the bumper 119—at the distal end of the vial assembly 110—to the patient's skin surface. The needle 118 extends beyond the distal end of the bumper 119 by a target needle insertion depth (e.g., between 0.040″ and 0.060″) such that depression of the bumper 119 onto the skin surface both: locates the needle 118 at this target insertion depth into the dermis; and increases locate hydrostatic pressure within the dermis around the needle 118, thereby inducing flow of interstitial fluid into the needle 118.

As shown in FIG. 7, a user may: strap the base assembly 120 to a patient's body (e.g., arm, leg); assemble the vial assembly 110 as described above; insert the vial assembly 110 into the base 121 aperture in the base 121, thereby setting the distal end of the bumper 119 against the skin surface and inserting the needle 118 into the patient's dermis; depress the vial assembly 110 downward against the patient's skin with a preferred force (or pressure) (e.g., with the user's forefinger); and then depress (or withdraw) the lock ring 125 to lock the vial assembly 110 to the base 121 (e.g., with the user's thumb and/or middle finger). Accordingly, the base assembly 120 and the strap 130 can cooperate to maintain application of the vial assembly 110 against the patient's skin surface with (approximately) this preferred force (or pressure).

Therefore, the system can enable: consistent application of force by the bumper 119 against a skin surface at a sample site on a patient; consistent increase in hydrostatic pressure within the dermis during collection of a sample; and hands-free sample collection once a user sets pressure of the of the bumper 119 against the skin surface (E.g., by depressing the proximal end of the via with her forefinger) and locates the lock ring 125 in the he locked position (e.g., by depressing the lock ring 125 with her thumb on the same hand).

More specifically, when the bumper 119 contacts the surface of the skin a the sample site, the bumper 119 can: deform inwardly against the skin; and depress skin at the sample site circumferentially around the needle 118, thereby forming a bulge and/or an adjacent valley in the skin and increasing local hydrostatic pressure within the interstitial volume in dermis adjacent the distal end of the needle 118. This increase in local hydrostatic pressure thus generated by the bumper 119 may then drive interstitial fluid from the dermis, through the needle 118, and into the vial 112.

### 6.1 Interstitial Fluid Sample Collection

To collect a sample of interstitial fluid from a sample site on a patient, a user (e.g., a technician, a physician) may: strap the base assembly 120 to a forearm (or to another extremity, to a buttocks, to a torso); unpackage the vial 112 and the collar 114—including a diaphragm seal 113 extending across an open end of the vial 112—from sterile packaging; unpackage the needle 118 and the needle casing 116 from sterile packaging; screw the needle casing 116 onto the collar 114, driving the proximal end of the needle 118 toward the vial 112 to puncture the diaphragm seal 113; unpackage the bumper 119 with the sterile sleeve 111—located in the aperture of the bumper 119—from sterile packaging; insert the needle 118 into the sterile sleeve 111 to locate the needle 118 through the bumper 119; and seat the bumper 119 around the needle 118 head, as shown in FIG. 6; and remove and discard the sterile sleeve 111.

Thus, the bumper 119 is located around, centered about, and remains out of contact with the needle 118 such that the distal end of the needle 118 remains sterile prior to contact with a patient's skin.

The user may then: secure the base assembly 120 to the user's arm (or leg, buttocks, abdomen) wrapping the strap 130 (e.g., an elastic band, a hook-and-loop strap) around the patient's arm such that the aperture is located over a sample site on the patient; release the lock ring 125 to the unlocked position; insert the vial assembly 110 into the aperture; and depress the top of the vial 112 downwardly into the aperture to bring the bumper 119 into contact with the patient's skin and to insert the needle 118 into the patient's dermis. While depressing the vial 112 downwardly toward the patient's skin with a preferred or target force, the user may then depress the lock ring 125, which drives the set of internal retention features 124 into contact with the set of external retention features 115 and thus retains the vial assembly 110 at this position within the base assembly 120 and maintains application of this force (approximately) against the skin surface by the bumper 119 of the vial assembly 110.

Then, in response to an insufficient flow rate or volume of interstitial fluid, the user may: release the lock ring 125; depress the vial assembly 110 further into the aperture of the base assembly 120 to increase the force of the bumper 119 on skin surface; and depress the lock ring 125 to lock the vial assembly 110 into place.

Additionally or alternatively, if the patient indicates perception of pain (e.g., vocally, by wincing, by tensing), the user may: remove the base assembly 120 from the patient; relocate the base assembly 120 to a second location on the patient's body; reassemble a second vial assembly 110 with a new, sterile needle; and reinsert the vial assembly 110 into the base assembly 120 to locate the needle 118 over a second skin surface proximal the second location.

### 6.2 Vial Assembly Retention

In one implementation shown in FIGS. 2 and 3, the collar 114 includes the set of external retention features 115 including: a first annular rib arranged on the collar 114 and extending radially outwardly from the collar 114; a second annular rib arranged on the collar 114, extending radially outwardly from the collar 114, and offset above the first annular rib; and a third annular rib arranged on the collar 114; extending radially outwardly from collar 114, and offset above the second annular rib. In this implementation, the lock ring 125 is configured: to compress the set of internal retention features 124 inwardly toward the base 121 aperture in order to locate an each internal retention feature 124 between the first annular rib and the second annular rib, thereby retaining a distal end of the needle 118 at a first depth below the base 121; and to compress the set of internal retention features 124 inwardly toward the base 121 aperture in order to locate each internal retention feature 124 between the second annular rib and the third annular rib, thereby retaining the distal end of the needle 118 at a second depth, less than the first depth, below the base 121.

More specifically, in this implementation the collar 114 includes a set of annular ribs: arranged on the collar 114; defining a vertical offset distance between each annular rib in the set of annular ribs; extending radially from the collar 114; and configured to sit on an internal retention feature 124 of the column 123 in the base assembly 120. In this implementation the set of annular ribs can define a range of vertical positions of the vial assembly 110 within the aperture and that correspond to a range of compressive forces of the bumper 119 against the skin surface. For example, in this implementation: the first annular rib can define a first offset distance below the second annular rib between 0.010″ and 0.050″ (e.g., 0.030″); and the second annular rib can define a second offset distance below the third annular rib between 0.010″ and 0.050″ (e.g., 0.030″). Accordingly, the system can enable the user to set the depth of the vial assembly 110—on the base assembly 120—over a series of depths stepped by a distance of between 0.010″ and 0.050″.

Therefore, the user may control force applied by the system to the sample site on via the bumper 119 by setting the inner retention features on the base 121 against a select external retention feature 115 (or within a valley defined by two adjacent external retention features 115) on the collar 114 of the vial assembly 110.

### 7. ANGULAR OFFSET AND DEPTH OF VIAL ASSEMBLY

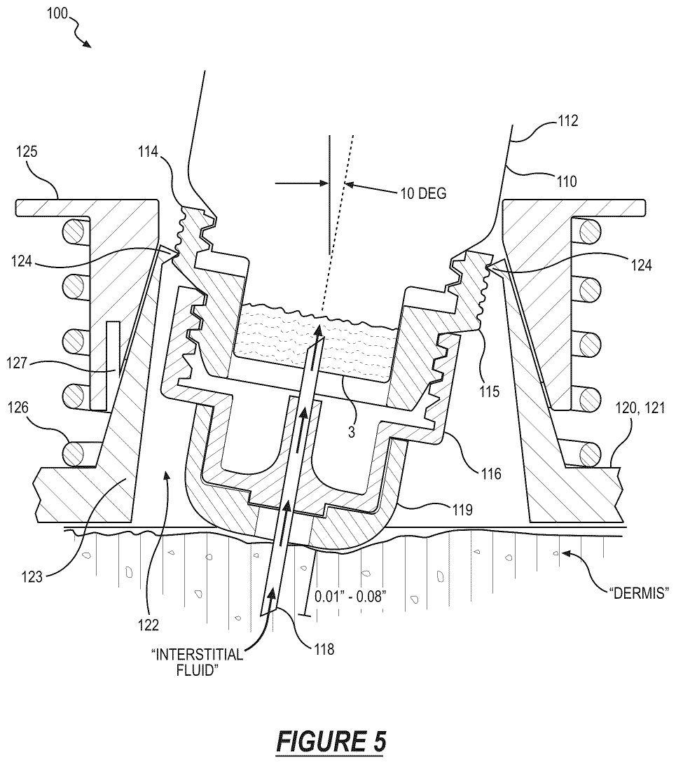

In one variation shown in FIG. 5, the base assembly 120 is configured to locate the vial assembly 110 with the axis of the via offset from the axis of the base 121 aperture (or “pitched”), such as by an angle between 0° (i.e., normal to the skin surface) and 10°.

In one implementation described above, the collar 114 of the vial assembly 110 defines a vertical array of annular ribs. The inner diameter of the base 121 aperture is greater than the outer diameter of the vial assembly 110. The base assembly 120 includes three internal retention features 124 configured to engages the same or different annular ribs on the collar 114. For example, the annular ribs can be vertically offset on the collar 114 by a pitch distance such that: the internal retention features 124 on the base assembly 120 engage a single valley between two annual ribs to locate the vial assembly 110 coaxial with the base 121 aperture; a first internal retention feature 124 and a second the internal retention feature 124 engage a first valley between two annual ribs and a third internal retention feature 124 engages a second valley below (or above) the first valley to locate the vial 112 at a first pitch angle of 2° within base aperture; and the first internal retention feature 124 and the second the internal retention feature 124 engage the first valley and the third internal retention feature 124 engages a third valley below (or above) the second valley to locate the vial 112 at a second pitch angle of 4° within base aperture; etc.

The lock ring 125 can selectively lock the internal retention features 124 against these external retention features 115 accordingly in order to control both the vertical position and the pitch angle of the vial assembly 110 within the base 121 aperture.

Thus, when the vial assembly 110 is pitched within the far, the needle 118 can enter this skin at a corresponding angle, and the circumferential edge of the bumper 119—surrounding the needle 118—can apply a non-uniform force (or pressure) against the skin surface about the needle 118. Such non-uniform force (or pressure) may yield non-uniform increases in hydrostatic pressure around the needle 118. Such non-uniform increases in hydrostatic pressure around the needle 118 may induce fluid flow upwardly through the need and into the vial 112 with less inhibition of interstitial fluid flow within the dermis toward the needle 118, thereby increasing total volume flow rate of interstitial fluid into the vial 112.

### 8. FLUID CONTACT/FLUID DRAW FEEDBACK

In one variation shown in FIG. 4, the system includes an electrical circuit configured to detect presence of interstitial fluid in the vial 112 and/or to detect flow of interstitial fluid into the vial 112 and to indicate such presence of flow to the user.

In one implementation, the vial 112 includes a conductive (e.g., silver) plating extending continuously from an external surface of the vial 112 to an internal surface of the vial 112 proximal an opening (or “mouth”) of the vial 112. The system includes: a battery (e.g., a coin cell battery) located on the base 121; light element (e.g., LED) coupled to a first (e.g., positive) terminal of the battery and located on the base 121; a vial 112 contactor (e.g., a spring-loaded tag or pin) located within the column 123 of the base 121, configured to contact the plated external surface of the vial 112, and electrically coupled to the light element opposite the battery (e.g., via a conductive via or flexible PCB; and a skin conductor coupled to a second (e.g., negative) terminal of the battery, located on an interior surface of the base 121, and configured to contact the skin surface of a patient. Thus, in this implementation, when a volume of interstitial fluid flows up the needle 118 and into the vial 112, the interstitial fluid—which contains ions—may bridge a gap between the (conductive) needle and the plated internal surface of the vial 112. This volume of interstitial fluid this close an electrical circuit and enables electrons to flow: from the battery; through the skin conductor; into the patient's skin; into the needle 118; through interstitial fluid bridge a gap between the (conductive) needle and the plated internal surface of the vial 112 via this volume of interstitial fluid; into the plated internal surface of the vial 112; to the plated external surface of the vial 112; through the vial 112 contactor; to the light element; and back to the battery, thereby illuminating the light element and visually indicating to the user than interstitial fluid is flowing into the vial 112.

Therefore, in this variation, a small volume of interstitial fluid collected within the vial 112 can close an electrical circuit, which actives a light element (or vibrator or other visual or haptic indicator) that indicates presence or flow of interstitial fluid into the vial 112. Thus, if the user inserts the vial assembly 110 into the base 121 aperture, depresses the vial assembly 110 with an initial target pressure, and closed the lock ring 125 but sees no activation of the light element within a nominal time period (e.g., five seconds, fifteen seconds), the user may release the lock ring 125, further depress the vial assembly 110 against the user's skin surface with a greater force, and then re-lock lock ring 125, which may thus induce interstitial fluid to flow into the vial 112 with no or minimal increase in perceived pain by the patient.

### 9. VARIATION: DOSING CARTRIDGE

In one variation, the system further includes a vial 112 containing a sample (e.g., a medication) for dosing into the dermis of a user. This sample may then be absorbed into interstitial fluid, which may then transport the sample into the patient's circulatory.

In one implementation, the vial 112 is elastic and may be compressed (or “squeezed”) by a user to displace contents of the vial 112, through the needle 118, and into a patient's dermis where these contents diffuse into the user's interstitial fluid, which may carries these contents to the user's circulatory system.

In another implementation, the vial 112 includes a barrel and plunger. In this implementation, a user may depress the plunger to displace contents of the vial 112, through the needle 118, and into a patient's dermis where these contents diffuse into the user's interstitial fluid, which may carries these contents to the user's circulatory system. Furthermore, in this implementation, the base 121 may carry a force—applied by the user to depress the plunger—across the user's broader skin surface in order to prevent local increase in pressure applied by the bumper 119 and to prevent further insertion of the needle 118 into the patient's skin, thereby yielding no or minimal increase in perception of pain by the patient as contents of the vial 112 are emptied into the patient's dermis.

Therefore, the system can be configured to: extract interstitial fluid from a patient's dermis; and to deliver medication, supplements, and/or other materials into the circulatory system of the patient with minimal or no perception of pain.

### 10. VARIATION: FRICTION-BASED VIAL RETENTION

In one variation, a system includes: a vial assembly 110 including a vial 112, a needle 118 coupled to a distal end of the vial 112, and a bumper 119 seated around the distal end of the needle 118 (and excluding a collar 114); and a base assembly 120. The base assembly 120 includes: a base plate defining a base aperture configured to receive the vial assembly 110; a column 123 extending upwardly from the base 121 around the aperture; and retention features located around the base 121 aperture and configured to directly engage and retain a smooth external surface on the vial 112—rather than external retention features 115 of a collar 114; and a lock ring 125 operable in a locked position to drive the internal retention features 124 inwardly toward the base 121 aperture to engage and mate against (or “squeeze”) the vial 112. In this variation, the retention features can define a set of friction surfaces (e.g., rubberized tips) configured to hold the vial assembly 110 at a particular vertical position within the base 121 aperture.

Therefore, in this variation, the vial assembly 110 can exclude a collar 114 and/or external retention features 115, and the internal retention features 124 can directly mate with a smooth external surface of the vial 112 to retain the vial assembly 110 over a continuous range first vertical positions within the base 121 aperture.

### 11. VARIATION: INVERTED LOCK RING

In one variation, the lock ring 125 can be configured to transition from the unlocked position to the locked position by lifting—rather than lowering—on the base 121, thereby enabling a user to set the applied force of the vial assembly 110 on the patient's skin surface and locking the lock ring 125 within a single hand and in a single motion, that is: depressing the proximal end of the vial assembly 110 with a thumb; and lifting the lock ring 125 with her middle finger and forefinger on the same hand. For example, in this variation, the internal and external cam surfaces of the lock ring 125 and the internal retention features 124 described above can be inverted.

Therefore, in this variation, the system can enable ergonomic one-handed use of the system.

As a person skilled in the art will recognize from the previous detailed description and from the figures and claims, modifications and changes can be made to the embodiments of the invention without departing from the scope of this invention as defined in the following claims.

I claim:

1. A system comprising:
a vial assembly comprising:
a vial:
configured to collect interstitial fluid;
a collar:
coupled to the vial; and
comprising a set of external retention features;
a needle casing:
coupled to the collar and enclosing the vial;
a needle:
extending from the needle casing; and
a bumper:
arranged on the needle casing opposite the vial; and
defining a needle aperture seated around and offset from the needle;
a base assembly:
comprising:
a base:
defining:
a base aperture configured to receive the vial assembly; and
a column extending above the base aperture and comprising a set of internal retention features configured to locate the set of external retention features; and
configured to locate over a skin surface of a patient;
a lock ring operable in an unlocked position and a locked position, the lock ring:
decoupled from the set of external retention features in the unlocked position; and
depressing the set of internal retention features inwardly toward the aperture to engage the set of external retention features of the vial assembly and to retain the vial assembly in a particular depth position in a range of depth positions within the base aperture, in the locked position; and
configured to depress the bumper of the vial assembly against the skin surface of the patient at a force corresponding to the particular depth position of the vial assembly within the base aperture; and
wherein the bumper applies the force to the skin surface of the patient, circumferentially about the needle, to create a first local hydrostatic pressure under the skin surface to drive interstitial fluid from below the skin surface, though the needle, and into the vial.

2. The system of claim 1:
wherein the bumper is configured to:
maintain the distal end of the needle at a depth between 0.001″ and 0.08″ below the skin surface.

3. The system of claim 1:
wherein the needle comprises a sterile hypodermic needle defining an outer diameter;
wherein the bumper:
defines an internal counterbore coaxial with the needle aperture; and
is configured to seat onto a distal end of the needle casing via a first interference fit; and
further comprising a sterile sleeve:
defining an inner diameter greater than the outer diameter;
configured to locate within the needle aperture of the bumper, via a second interference fit, during assembly of the bumper onto the needle casing to prevent contact between the bumper and the needle; and
removable from the bumper to expose the needle prior to insertion of the vial assembly into the base aperture of the base.

4. The system of claim 1:
further comprising a spring:
configured to bias the lock ring into the unlocked position; and
further comprising a ratchet mechanism:
configured to selectively retain the lock ring in the locked position.

5. The system of claim 1:
wherein the set of external retention features of the collar comprise:
a first annular rib:
arranged on the collar; and
extending radially outwardly from collar;
a second annular rib:
arranged on the collar;
extending radially outwardly from collar; and
offset above the first annular rib;
a third annular rib:
arranged on the collar;
extending radially outwardly from collar; and
offset above the second annular rib; and
wherein the lock ring is configured to:
displace the set of internal retention features inwardly toward the base aperture to locate an internal retention feature, in the set of internal retention features, between the first annular rib and the second annular rib to locate a distal end of the needle at a first depth below the base; and
compress the set of internal retention features to locate the internal retention feature, in the set of internal retention features, between the second annular rib and third annular rib to locate the distal end of the needle at a second depth, less than the first depth, below the base.

6. The system of claim 5:
wherein the first annular rib defines a first offset distance below the second annular rib between 0.010″ and 0.050″; and
wherein the second annular rib defines a second offset distance below the third annular rib between 0.010″ and 0.050″.

7. The system of claim 1:
wherein the collar defines a first outer diameter;
wherein the base aperture defines a second inner diameter greater than the first outer diameter;
wherein the vial assembly is configured to insert into the base aperture over a range of pitch angles between 0° and 10°; and
wherein the lock ring is configured to:
compress the set of internal retention features against the set of external retention features to retain the vial assembly at a pitch angle in the range of pitch angles.

8. The system of claim 1, wherein the bumper comprises:
an attachment surface configured to assemble onto a distal end of the needle casing;
a bell-shaped region extending distally from the attachment surface; and
an annular ridge:
arranged on a distal end of the bell-shaped region, encircling the needle aperture, offset above a distal end of the needle; and
defining a first point of contact between the bumper and the skin region.

9. The system of claim 8, wherein the bumper:
comprises an elastomeric material of a durometer between Shore 40 and Shore 80; and
is configured to deform against the skin surface during further insertion of the vial assembly into the base aperture of the base.

10. The system of claim 1:
wherein the vial comprises:
a diaphragm seal:
located across a distal end of vial; and
configured to seal the distal end of the vial; and
wherein the needle comprises:
a proximal end extending rearward from the needle casing and configured to puncture the diaphragm seal of the vial during assembly of the needle casing onto the collar.

11. The system of claim 1:
further comprising a strap:
arranged on the base; and
configured to retain the base on a forearm of the patient;
wherein the base:
locates the base aperture and the vial assembly over the forearm of the patient;
maintains the bumper of the vial assembly in contact with the skin surface of the patient;
wherein the bumper depresses against the skin surface to increase hydrostatic pressure, under the skin surface, to the first hydrostatic pressure greater than ambient air pressure; and
wherein the vial collects a volume of interstitial fluid driven through the needle via a difference between the first hydrostatic pressure and ambient air pressure.

12. A system comprising:
a base assembly:
comprising:
a base:
defining:
a base aperture configured to receive a vial assembly; and
a column extending above the base aperture and comprising a set of internal retention features configured to locate a set of external retention features of the vial assembly; and
configured to locate over a skin surface of a patient; and
a lock ring operable in an unlocked position and a locked position, the lock ring:
decoupled from the set of external retention features in the unlocked position; and
depressing the set of internal retention features inwardly toward the aperture, to engage the set of external retention features of the vial assembly and to retain the vial assembly in a particular depth position in a range of depth positions within the base aperture, in the locked position; and
configured to depress a bumper of the vial assembly against the skin surface of the patient at a force corresponding to the particular depth position of the vial assembly within the base aperture, the bumper of the vial assembly:
configured to apply the force to the skin surface of the patient, circumferentially about the needle, to create local hydrostatic pressure under the skin surface to drive interstitial fluid from below the skin surface, though the needle, and into the vial.

13. The system of claim 12, further comprising:
the vial assembly comprising:
a vial:
configured to collect interstitial fluid;
a collar:
coupled to the vial; and
comprising a set of external retention features configured to locate the set of internal retention features of the column;
a needle casing:
coupled to the collar and enclosing the vial;
a needle:
extending from the needle casing; and
the bumper:
arranged on the needle casing opposite the vial; and
defining a needle aperture seated around and offset from the needle.

14. The system of claim 12:
wherein the base defines a V-shaped surface configured to locate over an extremity of the patient and to contact the skin surface of the patient;
wherein the column defines the base aperture, the base aperture defining:
an angular pitch offset between 1° and 10° from the plane; and
wherein the lock ring is configured to:
compress the set of internal retention features against the set of external retention features to retain the vial assembly at the angular pitch offset.

15. The system of claim 12, further comprising:
a spring:
configured to bias the lock ring into the unlocked position; and
a ratchet mechanism:
coupled to the spring; and
configured to selectively retain the lock ring in the locked position.

16. A system comprising:
a vial assembly comprising:
a vial:
configured to collect interstitial fluid;
a collar:
coupled to the vial; and
comprising a set of external retention features configured to locate a set of internal retention features of a base assembly;
a needle casing:
coupled to the collar and enclosing the vial;
a needle:
extending from the needle casing; and
a bumper:
arranged on the needle casing opposite the vial;
defining a needle aperture seated around and offset from the needle; and
configured to apply a force to a skin surface of a patient, circumferentially about the needle, to create local hydrostatic pressure under the skin surface to drive interstitial fluid from below the skin surface, though the needle, and into the vial.

17. The system of claim 16, further comprising:
a base assembly:
comprising:
a base:
defining:
a base aperture configured to receive the vial assembly; and
a column extending above the base aperture and comprising a set of internal retention features configured to locate the set of external retention features of the collar of the vial assembly; and
configured to locate over a skin surface of a patient;
a lock ring operable in an unlocked position and a locked position, the lock ring:
decoupled from the set of external retention features in the unlocked position; and
depressing the set of internal retention features inwardly toward the aperture, to engage the set of external retention features of the vial assembly and to retain the vial assembly in a particular depth position in a range of depth positions within the base aperture, in the locked position; and
configured to depress the bumper of the vial assembly against the skin surface of the patient at a force corresponding to the particular depth position of the vial assembly within the base aperture.

18. The system of claim 17:
wherein the collar comprises the set of external retention features comprising:
an external helical thread configured to engage an internal helical thread of the column to locate the vial assembly over a range of depths in the column; and
wherein the bumper is configured to:
interface with the skin surface of the patient; and
rotate about the needle and remain in a fixed angular position on the skin surface as the external helical thread of the collar engages the internal helical thread of the column.

19. The system of claim 16:
wherein the collar comprises the set of external retention features comprising:
a first annular rib:
arranged on the collar; and
extending radially outwardly from collar;
a second annular rib:
arranged on the collar;
extending radially outwardly from collar; and
offset above the first annular rib; and
a third annular rib:
arranged on the collar;
extending radially outwardly from collar; and
offset above the second annular rib; and
further comprising a lock ring:
located on a base assembly configured to accept the vial assembly; and
configured to:
compress the set of internal retention features to locate an internal retention feature, in the set of internal retention features, between the first annular rib and the second annular rib to locate a distal end of the needle at a first depth below the base; and
compress the set of internal retention features to locate the internal retention feature, in the set of internal retention features, between the second annular rib and the third annular rib to locate the distal end of the needle at a second depth, less than the first depth, below the base.

20. The system of claim 16:
wherein the needle comprises a sterile hypodermic needle defining an outer diameter;
wherein the needle casing:
defines an internal counterbore coaxial with the needle aperture; and
is configured to seat onto a distal end of the needle casing via a first interference fit; and
further comprising a sterile sleeve:
defining an inner diameter greater than the outer diameter;
configured to locate within the needle aperture of the bumper, via a second interference fit, during assembly of the bumper onto the needle casing to prevent contact between the bumper and the needle; and
removable from the bumper to expose the needle prior to insertion of the vial assembly into the base aperture of the base.
